(19) United States
(12) Patent Application Publication
(10) Pub. No.: US 2025/0369903 A1
(43) Pub. Date: Dec. 4, 2025

(54) HIGH-TEMPERATURE AND HIGH-PRESSURE CORE DISPLACEMENT TEST SYSTEM AND METHOD

(71) Applicant: INSTITUTE OF ROCK AND SOIL MECHANICS, CHINESE ACADEMY OF SCIENCES, WUHAN (CN)

(72) Inventors: Qiang XUE, WUHAN (CN); Zhixiang CHEN, WUHAN (CN); Yong WAN, WUHAN (CN); Yan WANG, WUHAN (CN); Quan JIANG, WUHAN (CN)

(73) Assignee: INSTITUTE OF ROCK AND SOIL MECHANICS, CHINESE ACADEMY OF SCIENCES, WUHAN (CN)

(21) Appl. No.: 19/296,153

(22) Filed: Aug. 11, 2025

(30) Foreign Application Priority Data
Aug. 19, 2024 (CN) 202411134278.8

(51) Int. Cl. G01N 23/046; G01L 1/22; G01L 1/24; G01N 15/00; G01N 15/08; G01N 33/24

(52) U.S. Cl. CPC G01N 23/046; G01L 1/2293; G01L 1/243; G01N 15/082; G01N 33/246; G01N 2015/0034; G01N 2223/309; G01N 2223/3106; G01N 2223/311; G01N 2223/419; G01N 2223/616

(57) ABSTRACT

A high-temperature and high-pressure core displacement test system includes a CT scanner configured to perform CT scanning, a gripper system configured to grip a core, a machine tool configured to move the gripper system to the CT scanner for CT scanning, a gripper load control system configured to heat and input a fluid into or/and receive a fluid output from the gripper system, and an acquisition and data analysis system configured to acquire a temperature and a pressure within a core gripping cavity, and detection data of the CT scanner and strain rosettes for analysis. According to the high-temperature and high-pressure core displacement test system and method, the strength of the gripper system in a high-temperature and high-pressure environment is improved, the projection performance of CT rays is ensured, and the optical fiber positioning precision and the core deformation measurement precision in a CT rapid scanning state are improved.

### CROSS-REFERENCE TO RELATED APPLICATION

This application claims priority to Chinese Patent Application No. 202411134278.8 with a filing date of Aug. 19, 2024. The content of the aforementioned application, including any intervening amendments thereto, is incorporated herein by reference.

### FIELD OF THE INVENTION

The present application relates to the field of testing by physical properties of materials, in particular to a high-temperature and high-pressure core displacement test system and method.

### BACKGROUND OF THE INVENTION

Behaviors such as movement and displacement of a fluid inside a rock in the process of deep resource development cause deformation or micro-cracking of a solid structure of the rock, so that engineering problems such as local compaction, uneven fracture networks, and pore gas blockage-induced bursting occur, which affects the energy efficiency of resource development. A core displacement computed tomography (CT) test is a cutting-edge method for obtaining the coupling characteristics of medium flow and deformation in cores during rock seepage or energy displacement production. Conducting the core displacement CT test has an important role in reasonably quantifying the local deformation and pore fluid migration characteristics within the cores during the displacement process and improving the accuracy of simulation predictions for deep space and resource development processes.

At present, the core displacement CT test needs to solve the following problems: 1. whether a CT test speed can be matched with a seepage process to avoid image blurring due to core deformation or fluid migration during scanning; 2. whether small seepage deformation of the core can be captured by CT; 3. the influence of thermal radiation generated by long-term heating of a sample on CT equipment is counteracted, and the uniformity of a temperature field of a rock sample is realized; and 4. whether a test environment meets the low density requirements of materials used in an external environment of a sample required for CT projection. The above problems directly restrict the test accuracy of the core displacement CT test, making fast scanning of the test process impossible with the existing XCT (a scanning speed can reach several or even dozens of hours), and the problem of scanned image blurring is prone to occur; using medical CT can achieve fast scanning but cannot accurately describe the overall deformation of the rock sample; the thermal radiation generated by in-situ electric heating and confining pressure heating schemes easily affects the test of scanning equipment, and there is a practical problem of a large temperature gradient at different points of the sample.

### SUMMARY OF THE INVENTION

An object of the present application is to provide a high-temperature and high-pressure core displacement test system and method, which improves the strength of a gripper system in a high-temperature and high-pressure environment and ensures the projection performance of CT rays, improving the optical fiber positioning precision and the core deformation measurement precision in a CT rapid scanning state, and realizing correction and measurement calculation of the overall volume change of a core by measuring the expansion volume change of the gripper system, improving the stability and precision of a core displacement CT test under high-temperature and high-pressure conditions.

The present application is implemented as follows:
the present application provides a high-temperature and high-pressure core displacement test system, including:
a CT scanner, configured to perform CT scanning;
a gripper system, including a base, a gripping container and an end cover which are connected in sequence, the base, the gripping container and the end cover enclosing a core gripping cavity containing a core gripping module, wherein the base is provided with an osmotic pressure inlet hole, an optical fiber inlet hole, an optical fiber outlet hole, and an annular pressure inlet hole and an annular pressure outlet hole which respectively communicate with the core gripping cavity; the gripping container includes a carbon fiber wound polyether-ether-ketone (PEEK) sleeve sleeving the core gripping module, alloy flange ends respectively bonded to both ends of the carbon fiber wound PEEK sleeve, a plurality of strain rosettes disposed on an outer wall of the carbon fiber wound PEEK sleeve, and a carbon powder heating film sleeving outer sides of the strain rosettes, and the two alloy flange ends are respectively connected with the base and the end cover; the core gripping module includes water permeable plates respectively clamped to two ends of a cylindrical core, a sample cap pressing against the end cover, a stiffness-limited optical fiber wound around an outer wall of the core, and a heat shrinkable sleeve sleeving an outer side of the stiffness-limited optical fiber, the two water permeable plates respectively abut against the base and the sample cap, the sample cap is provided with an osmotic pressure outlet hole, the osmotic pressure inlet hole and the osmotic pressure outlet hole respectively communicate with the two water permeable plates, and two ends of the stiffness-limited optical fiber respectively penetrate through the optical fiber inlet hole and the optical fiber outlet hole; and a pressure-resistant valve is connected to the annular pressure outlet hole;
a machine tool, configured to move the gripper system to the CT scanner for CT scanning of a core or move the gripper system out of the CT scanner;
a gripper load control system, configured to heat and input a fluid into the osmotic pressure inlet hole and the annular pressure inlet hole and receive a fluid output from the osmotic pressure outlet hole; and
an acquisition and data analysis system, configured to acquire a temperature and a pressure within the core gripping cavity, and detection data of the CT scanner and the strain rosettes for analysis.

In some optional embodiments, the base includes a first step, a second step, a third step and a fourth step which are connected in sequence, the annular pressure inlet hole and the annular pressure outlet hole penetrate through the first step, the second step and the third step, and the fourth step abuts against the water permeable plate; and a peripheral wall of the third step and a peripheral wall of the fourth step are each provided with at least one perfluoroether high-temperature-resistant sealing ring.

In some optional embodiments, the annular pressure inlet hole is connected with a pressure-resistant tube located inside the core gripping cavity, and a length of the pressure-resistant tube is 0.6-0.9 times a height of the core.

In some optional embodiments, the sample cap includes a fifth step and a sixth step which are connected in sequence, the fifth step presses against the water permeable plate, and a peripheral wall of the sixth step is provided with at least one perfluoroether high-temperature-resistant sealing ring.

In some optional embodiments, the stiffness-limited optical fiber includes a bare optical fiber and optical fiber sleeves nested and fixed to the bare optical fiber at intervals, a spacing between centers of two of the optical fiber sleeves being 0.65 D, D being a positive integer.

In some optional embodiments, each strain rosette is of a three-piece right angle shape, and a resistance wire of each strain rosette is made of silicon; and data cables in the strain rosettes are composed of thin film graphene or black phosphorene strips, and the graphene or black phosphorene strips have a width of 0.25-0.26 mm and a thickness of 0.8-1.2 mm.

In some optional embodiments, the gripper load control system includes a confining pressure plunger pump connected to the annular pressure inlet hole, an upstream high-temperature heating repeater connected to the osmotic pressure inlet hole and a downstream high-temperature heating repeater connected to the osmotic pressure outlet hole, the upstream high-temperature heating repeater is connected with an upper head plunger pump, and the downstream high-temperature heating repeater is connected with a lower head plunger pump.

In some optional embodiments, the system further includes a heat-insulating jacket wrapping the base, the gripping container, and the end cover.

The present application also provides a high-temperature and high-pressure core displacement test method, performed by using the high-temperature and high-pressure core displacement test system described above, and including the steps of:
Step 1, controlling the gripper load control system to input a heating fluid into the osmotic pressure inlet hole, closing the pressure-resistant valve when a fluid continuously flows out of the annular pressure outlet hole, and adopting a core temperature field control method based on a temperature gradient to circulate a fluid inside the core gripping cavity to uniformly distribute the temperature of a core; and
Step 2, calculating a total volume change V_{Di} of the core at a moment i according to the following formula:

$${V}_{Di} = {V}_{i} - Δ {V}_{F i} ;$$

wherein V_{Di} is the total volume change of the core at the moment i; and Vᵢ is a liquid inlet amount of the annular pressure inlet hole at the moment i; and calculating the amount of expansion deformation ΔV_{Fi} of the gripper system at the moment i by using the following formula:

$$Δ {V}_{Fi} = {\frac{1}{64 π} [ {\sum }_{k = 1}^{4} {R}_{ki} \cdot ( 1 + {L}_{0} ) ]}^{2} \cdot \frac{1}{3} {\sum }_{k = 1}^{3} {V}_{ki} \cdot ( 1 \to {V}_{0} ) - \frac{{L}_{0}^{2} {V}_{0}}{4 π} ;$$

wherein π is a ratio of a circumference of a circle to its diameter; Rₖᵢ and Vₖᵢ are a radial strain and an axial strain detected by a strain rosette k at the moment i, respectively; and L₀ and V₀ are a radial circumference and an axial height of the gripping container, respectively.

In some optional embodiments, the core temperature field control method based on a temperature gradient includes the steps of:
Step 1.1, connecting the annular pressure inlet hole to the confining pressure plunger pump through the upstream high-temperature heating repeater; connecting the annular pressure outlet hole to the lower head plunger pump through the downstream high-temperature heating repeater; setting the temperature of the upstream high-temperature heating repeater and the downstream high-temperature heating repeater to be Tᵢ₊ₖ, setting the heating time to be tᵢ₊ₖ, and opening the pressure-resistant valve to maintain the temperature of a fluid inside the gripper system to be Tᵢ₊ₖ to heat the core until the heating time reaches tᵢ₊ₖ by sequentially performing injection through the confining pressure plunger pump and sucking out the fluid through the lower head plunger pump, performing injection through the lower head plunger pump and sucking out the fluid through the confining pressure plunger pump;
Step 1.2, setting a heating temperature of the upstream high-temperature heating repeater and the downstream high-temperature heating repeater to be Tᵢ₊ⱼ, Tᵢ₊ⱼ<Tᵢ₊ₖ, setting the heating time to be tᵢ₊ⱼ, and adopting the method in Step 1 to continue heating the core until the heating time reaches tᵢ₊ⱼ;
Step 1.3, setting a heating temperature of the upstream high-temperature heating repeater and the downstream high-temperature heating repeater to be Tᵢ, Tᵢ₊ⱼ<Tᵢ<Tᵢ₊ₖ, setting the heating time to be tᵢ, and adopting the method in Step 1 to continue heating the core until the heating time reaches tᵢ; and
Step 1.4, closing the downstream high-temperature heating repeater, and closing the pressure-resistant valve between the annular pressure outlet hole and the lower head plunger pump, connecting the lower head plunger pump with the osmotic pressure outlet hole, and connecting the upper head plunger pump with the osmotic pressure inlet hole; and then setting a heating temperature of the carbon powder heating film to be Tᵢ, Tᵢ₊ⱼ<Tᵢ<Tᵢ₊ₖ, and continuing heating the core until the end of a test.

The beneficial effects of the present application are as follows: the high-temperature and high-pressure core displacement test system provided in the present application includes the CT scanner configured to perform CT scanning, the gripper system configured to grip the core, the machine tool configured to move the gripper system to the CT scanner for CT scanning, the gripper load control system configured to heat and input a fluid into or/and receive a fluid output from the gripper system, and the acquisition and data analysis system configured to acquire the temperature and the pressure within the core gripping cavity, and detection data of the CT scanner and the strain rosettes for analysis; the gripper system includes the base, the gripping container and the end cover which are connected in sequence, the base, the gripping container and the end cover enclosing the core gripping cavity containing the core gripping module; the base is provided with the osmotic pressure inlet hole, the optical fiber inlet hole, the optical fiber outlet hole, and the annular pressure inlet hole and the annular pressure outlet hole which respectively communicate with the core gripping cavity; the gripping container includes the carbon fiber wound PEEK sleeve sleeving the core gripping module, the alloy flange ends respectively bonded to both ends of the carbon fiber wound PEEK sleeve, the plurality of strain rosettes disposed on the outer wall of the carbon fiber wound PEEK sleeve, and the carbon powder heating film sleeving the outer sides of the strain rosettes, and the two alloy flange ends are respectively connected with the base and the end cover; the core gripping module includes the water permeable plates respectively clamped to two ends of the cylindrical core, the sample cap pressing against the end cover, the stiffness-limited optical fiber wound around the outer wall of the core, and the heat shrinkable sleeve sleeving the outer side of the stiffness-limited optical fiber, the two water permeable plates respectively abut against the base and the sample cap, the sample cap is provided with the osmotic pressure outlet hole, the osmotic pressure inlet hole and the osmotic pressure outlet hole respectively communicate with the two water permeable plates, and two ends of the stiffness-limited optical fiber respectively penetrate through the optical fiber inlet hole and the optical fiber outlet hole; and the pressure-resistant valve is connected to the annular pressure outlet hole. According to the high-temperature and high-pressure core displacement test system and method provided in the present application, the strength of the gripper system in a high-temperature and high-pressure environment is improved, the projection performance of CT rays is ensured, and the optical fiber positioning precision is improved by optical fiber confined deformation on the surface of a cylindrical sample, and the core deformation measurement precision in a CT rapid scanning state is improved; by measuring the expansion volume change of the gripper system, correction and measurement calculation of the overall volume change of the core are realized, and the stability and test precision of the core displacement CT test under high-temperature and high-pressure conditions are improved.

### BRIEF DESCRIPTION OF DRAWINGS

In order to illustrate the technical solutions of the embodiments of the present application more clearly, the drawings required to be used in the embodiments will be briefly described below, and it should be understood that the following drawings illustrate only certain embodiments of the present application and therefore should not be regarded as limiting the scope, and that other related drawings can also be obtained from these drawings without inventive steps for those of ordinary skill in the art.

FIG. 1 is a schematic structural diagram of a high-temperature and high-pressure core displacement test system according to an embodiment of the present disclosure;

FIG. 2 is a schematic cross-sectional structural diagram of a gripper system in the high-temperature and high-pressure core displacement test system according to the embodiment of the present disclosure;

FIG. 3 is a schematic structural diagram of a base of the gripper system in the high-temperature and high-pressure core displacement test system according to the embodiment of the present disclosure;

FIG. 4 is a schematic diagram of a partial structure of a gripping container omitting a carbon powder heating film in the high-temperature and high-pressure core displacement test system according to the embodiment of the present disclosure;

FIG. 5 is a schematic diagram of a partial structure of a stiffness-limited optical fiber of the high-temperature and high-pressure core displacement test system according to the embodiment of the present disclosure;

FIG. 6 is a flow chart showing a high-temperature and high-pressure core displacement test method according an embodiment of the present disclosure; and

FIG. 7 is a flow chart showing a core temperature field control method based on the temperature gradient.

In the drawings: 100, CT scanner; 200, gripper system; 210, base; 211, osmotic pressure inlet hole; 212, optical fiber inlet hole; 213, optical fiber outlet hole; 214, annular pressure inlet hole; 215, annular pressure outlet hole; 216, first step; 217, second step; 218, third step; 219, fourth step; 220, gripping container; 221, carbon fiber wound polyether-ether-ketone (PEEK) sleeve; 222, alloy flange end; 223, strain rosette; 224, carbon powder heating film; 230, end cover; 231, end cover body; 232, pressing end; 240, core gripping module; 241, water permeable plate; 242, sample cap; 243, stiffness-limited optical fiber; 244, heat shrinkable sleeve; 245, osmotic pressure outlet hole; 246, fifth step; 247, sixth step; 248, bare optical fiber; 249, optical fiber sleeve; 250, core gripping cavity; 251, temperature sensor opening; 252, pressure sensor opening; 260, pressure-resistant valve; 270, pressure-resistant tube; 280, heat-insulating jacket; 290, perfluoroether high-temperature-resistant sealing ring; 300, machine tool; 400, gripper load control system; 410, confining pressure plunger pump; 420, upstream high-temperature heating repeater; 430, downstream high-temperature heated repeater; 440, upper head plunger pump; 450, lower head plunger pump; 500, acquisition and data analysis system; and 600, core.

### DETAILED DESCRIPTION OF THE INVENTION

In order to make the objectives, technical solutions and advantages of the embodiments of the present application more clear, the technical solutions in the embodiments of the present application will be clearly and completely described below in conjunction with the accompanying drawings in the embodiments of the present application, and obviously, the described embodiments are part of the embodiments of the present application, rather than all of the embodiments. The components in the embodiments of the present application generally described and illustrated in the drawings herein may be arranged and designed in a variety of different configurations.

Thus, the following detailed description of the embodiments of the present application, as provided in the drawings, is not intended to limit the scope of the present application, as claimed, but is merely representative of the selected embodiments of the present application. Based on the embodiments in the present application, all other embodiments obtained by those of ordinary skill in the art without inventive step belong to the scope of protection of the present application.

It should be noted that like reference numerals and letters represent like items in the following figures, and therefore, once an item is defined in one figure, it needs not be further defined and explained in the subsequent figures.

In the description of the present application, it should be noted that the orientation or position relationship indicated by the terms such as “center”, “upper”, “lower”, “left”, “right”, “vertical”, “horizontal”, “inner”, and “outer” is based on the orientation or position relationship shown in the drawings, or the orientation or position relationship in which a product in the present application is conventionally placed during use, is merely for ease of description of the application and for simplicity of description, and is not intended to indicate or imply that the device or element referred to must have a particular orientation, and be constructed and operated in a particular orientation, and is therefore not to be construed as limiting the application. In addition, the terms such as “first,” “second,” and “third,” are used only to distinguish descriptions and are not to be construed as indicating or implying relative importance.

In addition, the terms such as “horizontal”, “vertical”, and “overhanging” do not mean that a component is required to be absolutely horizontal or overhung, but may be slightly inclined. For example, “horizontal” merely means that its orientation is more horizontal than “vertical”, and does not mean that the structure must be completely horizontal, but may be slightly inclined.

In the description of the present application, it should also be noted that unless expressly specified and limited otherwise, the terms “arranged”, “mounted”, “connected”, and “connection” should be broadly understood, for example, it can be fixed connection, detachable connection or integrated connection; it can be mechanical connection or electric connection; and it can be direct connection or indirect connection through an intermediate medium, and may be internal communication of two elements. For those of ordinary skill in the art, the specific meanings of the above terms in the present application may be understood according to specific situations.

In the present application, unless expressly specified and defined otherwise, a first feature being “above” or “below” a second feature may include that the first feature and the second feature are in direct contact or may include that the first feature and the second feature are not in direct contact but are in contact through another feature therebetween. Moreover, a first feature being “above”, “on” and “over” a second feature includes that the first feature is directly above and obliquely above the second feature, or simply indicates that the first feature is at a higher level than the second feature. A first feature being “below”, “under” and “beneath” a second feature includes that the first feature is directly below and obliquely below the second feature, or simply indicates that the first feature is at a level less than the second feature.

The features and properties of the high-temperature and high-pressure core displacement test system and method of the present application are described in further detail below with reference to the embodiments.

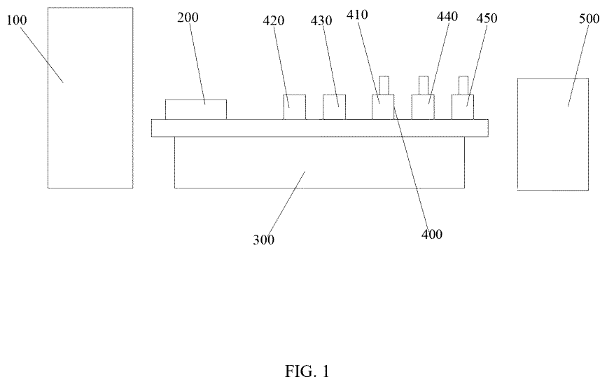

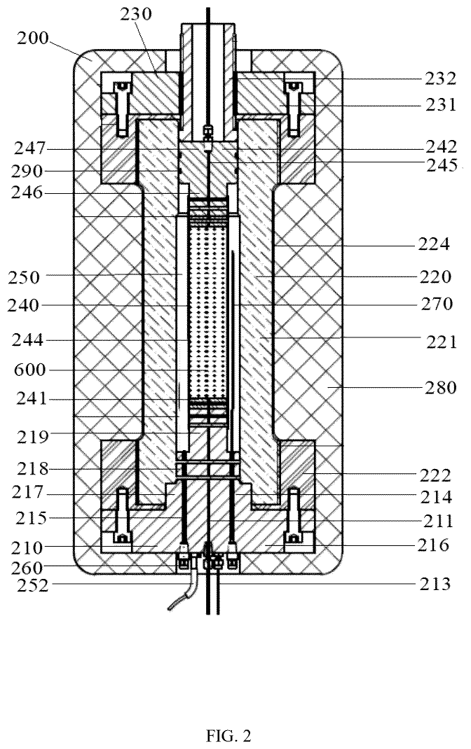

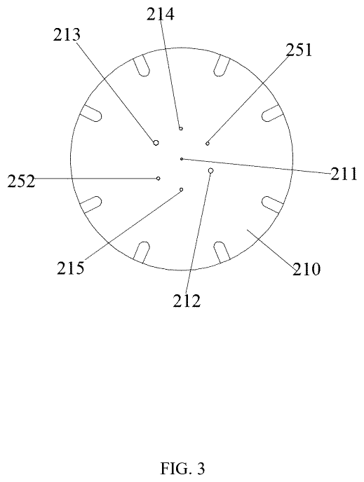

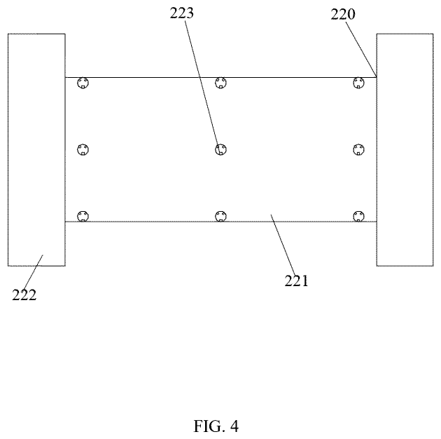

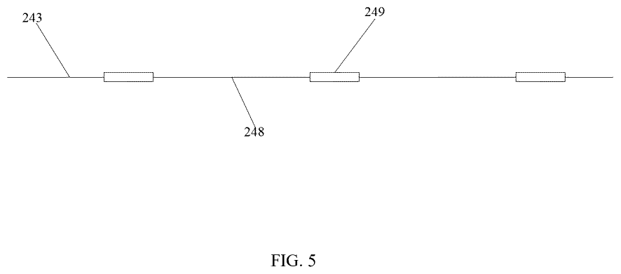

As shown in FIGS. 1, 2, 3, 4, and 5, an embodiment of the present application provides a high-temperature and high-pressure core displacement test system. The system is used for gripping a core 600 for a displacement test and includes a CT scanner 100 configured to perform CT scanning, a gripper system 200 configured to grip the core 600, a machine tool 300 configured to move the gripper system 200 to the CT scanner 100 for CT scanning of the core or move the gripper system 200 out of the CT scanner 100, a gripper load control system 400 configured to heat and input a fluid into an osmotic pressure inlet hole 211 and an annular pressure inlet hole 214 and receive a fluid output from an osmotic pressure outlet hole 245, and an acquisition and data analysis system 500 configured to acquire a temperature and a pressure within a core gripping cavity 250, and detection data of the CT scanner 100 and strain rosettes 223 for analysis;
the gripper system 200 includes a core gripping module 240, and a base 210, a gripping container 220 and an end cover 230 which are connected in sequence by bolts, and a heat-insulating jacket 280 sleeving the base 210, the gripping container 220 and the end cover 230, the base 210, the gripping container 220 and the end cover 230 enclosing the core gripping cavity 250 containing the core gripping module 240;

The base 210 includes a first step 216, a second step 217, a third step 218 and a fourth step 219 which are of a cylindrical shape and are connected in sequence, the base 210 is provided with an osmotic pressure inlet hole 211 passing through its center, and an optical fiber inlet hole 212, an optical fiber outlet hole 213, an annular pressure inlet hole 214, an annular pressure outlet hole 215, a temperature sensor opening 251 and a pressure sensor opening 252 which are arranged at intervals along its circumference, the optical fiber inlet hole 212, the optical fiber outlet hole 213, the annular pressure inlet hole 214, the annular pressure outlet hole 215, the temperature sensor opening 251 and the pressure sensor opening 252 all pass through the first step 216, the second step 217 and the third step 218 and communicate with the core gripping cavity 250, and a peripheral wall of the third step 218 and a peripheral wall of the fourth step 219 are each provided with two perfluoroether high-temperature-resistant sealing rings 290; an outer wall of the first step 216 is provided with eight circumferentially spaced bolt fixing positions; the annular pressure inlet hole 214 is connected with a pressure-resistant tube 270 located inside the core gripping cavity 250, the pressure-resistant tube 270 has a length of 0.8 times a height of the core 600, and the pressure-resistant tube 270 is a PEEK pressure-resistant tube; the annular pressure outlet hole 215 is connected with a pressure-resistant valve 260 which is a low-leakage-rate pressure-resistant valve; and the temperature sensor opening 251 and the pressure sensor opening 252 are correspondingly provided with a temperature sensor and a pressure sensor for temperature detection and pressure detection of a fluid inside the core gripping cavity 250; and

The gripping container 220 includes a carbon fiber wound polyether-ether-ketone (PEEK) sleeve 221 sleeving the core gripping module 240, alloy flange ends 222 respectively bonded to both ends of the carbon fiber wound PEEK sleeve 221, 12 arrayed strain rosettes 223 disposed on an outer wall of the carbon fiber wound PEEK sleeve 221, and a carbon powder heating film 224 sleeving outer sides of the strain rosettes 223, the two alloy flange ends 222 are respectively connected to the base 210 and the end cover 230 by bolts, and the two alloy flange ends 222 are each provided with bolt fixing positions arranged circumferentially at intervals; each strain rosette 223 is of a three-piece right angle shape, and a resistance wire of each strain rosette 223 is made of silicon; and data cables in the strain rosettes 223 are composed of thin film graphene, and graphene or black phosphorene strips have a width of 0.26 mm and a thickness of 0.1 mm.

The core gripping module 240 includes water permeable plates 241 respectively clamped to both ends of the core 600, a sample cap 242 pressing against the end cover 230, a stiffness-limited optical fiber 243 wound around an outer wall of the core 600, and a heat shrinkable sleeve 244 sleeving an outer side of the stiffness-limited optical fiber 243, the stiffness-limited optical fiber 243 includes a bare optical fiber 248 and optical fiber sleeves 249 nested and fixed to the bare optical fiber 248 at intervals, a spacing between centers of two optical fiber sleeves 249 being 0.65 D, D being a positive integer. The two water permeable plates 241 respectively abut against the fourth step 219 of the base 210 and the sample cap 242, a center of the sample cap 242 is provided with an osmotic pressure outlet hole 245, the osmotic pressure inlet hole 211 and the osmotic pressure outlet hole 245 respectively communicate with the two water permeable plates 241, and two ends of the stiffness-limited optical fiber 243 respectively penetrate through the optical fiber inlet hole 212 and the optical fiber outlet hole 213; and
the sample cap 242 includes a fifth step 246 and a sixth step 247 which are of a cylindrical shape and are connected in sequence, the fifth step 246 presses against the water permeable plate 241, the osmotic pressure outlet hole 245 penetrates through the fifth step 246 and the sixth step 247, and a peripheral wall of the sixth step 247 is provided with two perfluoroether high-temperature-resistant sealing rings 290. The end cover 230 includes an end cover body 231 provided with a threaded hole in a middle portion thereof, and a pressing end 232 threaded through and connected to the end cover body 231, an outer wall of the end cover body 231 is provided with eight circumferentially spaced bolt fixing positions, and the pressing end 232 presses against the sixth step 247.

The gripper load control system 400 includes a confining pressure plunger pump 410 connected to the annular pressure inlet hole 214, an upstream high-temperature heating repeater 420 connected to the osmotic pressure inlet hole 211 and a downstream high-temperature heating repeater 430 connected to the osmotic pressure outlet hole 245, an upper head plunger pump 440 is connected with the upstream high-temperature heating repeater 420, and a lower head plunger pump 450 is connected with the downstream high-temperature heating repeater 430; and a pipeline connected between the annular pressure inlet hole 214 and the confining pressure plunger pump 410 is provided with a flow meter. The acquisition and data analysis system 500 is electrically connected to the temperature sensor, the pressure sensor, the stiffness-limited optical fiber 243, the CT scanner 100, and the strain rosettes 223, respectively.

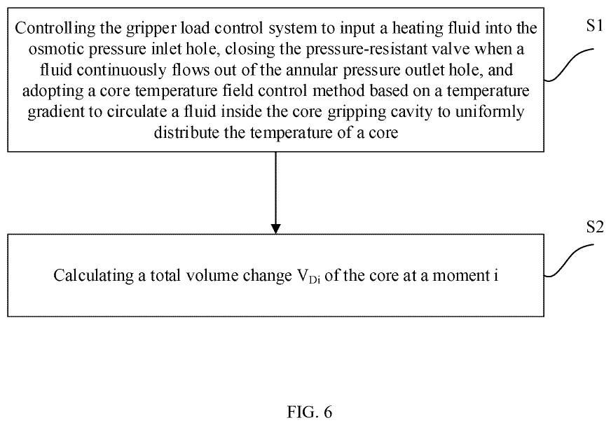

An embodiment of the present application also provides a high-temperature and high-pressure core displacement test method as shown in FIG. 6, performed by using the high-temperature and high-pressure core displacement test system described above, and including the following steps:
Step 0, the gripper system 200 is assembled; one water permeable plate 241 and the core 600 are placed on the fourth step 219 of the base 210 in sequence, the stiffness-limited optical fiber 243 is spirally wound and fixed onto the surface of the cylindrical core 600 after passing through the optical fiber inlet hole 212, and the stiffness-limited optical fiber 243 is led out through the optical fiber outlet hole 213; the core 600 and the fourth step 219 of the base 210 are sleeved with the heat shrinkable sleeve 244, and another water permeable plate 241 and the sample cap 242 are placed on an upper part of the core 600 in sequence; the heat shrinkable sleeve 244 is heated by a heat gun so that the heat shrinkable sleeve 244 sleeves and is tightly attached to the fourth step 219 of the base 210, the core 600, and the sample cap 242; the gripping container 220 with the arrayed strain rosettes 223 and the carbon powder heating film 224 adhered to an outer layer is in contact with the base 210 after passing through the core 600 connected to the heat shrinkable sleeve 244 from the end cover 230 to be fixed by bolts; and the pressing end 232 connected to the end cover body 231 is rotated and axially moved to clamp the core 600 between the base 210 and the end cover 230 to form the gripper system 200;
Step 1, the assembled gripper system 200 is inverted so that the base 210 faces upward and the end cover 230 faces downward, and a high-temperature confining pressure fluid is injected into the gripper system 200 by connecting the upper head plunger pump 440 to the osmotic pressure inlet hole 211 until the pressure-resistant valve 260 connected to the annular pressure outlet hole 215 is closed when the high-temperature confining pressure fluid continuously flows out from the annular pressure outlet hole 215; a core temperature field control method based on a temperature gradient is used to circulate the high-temperature confining pressure fluid within the core gripping cavity 250 to uniformly distribute the temperature of the core 600, the gripper system 200 is connected to the machine tool 300 by using a machine tool fastening frame, the temperature sensor and the pressure sensor are allowed to extend into the core gripping cavity 250 through the temperature sensor opening 251 and the pressure sensor opening 252, respectively, and the acquisition and data analysis system 500 is electrically connected to the temperature sensor, the pressure sensor, the stiffness-limited optical fiber 243, the CT scanner 100, and the strain rosettes 223, respectively.

The machine tool 300 is controlled to move the gripper system 200 to the CT scanner 100 for CT scanning, the CT scanner 100 is turned on, a liquid inlet amount Vᵢ of the annular pressure inlet hole 214 at a moment i is recorded, and a spatial position reading Dₖᵢ (xₖᵢ, yₖᵢ, zₖᵢ) of a measuring point determined by the stiffness-limited optical fiber 243, stress data Fₘᵢ (Rₖᵢ, Vₖᵢ) detected by the strain rosettes 223, a CT reading C_{Ki} (lₖᵢ, mₖᵢ, nₖᵢ) of an arrangement point of the stiffness-limited optical fiber 243, and a CT reading Cₘᵢ (lₘᵢ, mₘᵢ, nₘᵢ) of arrangement points of the strain rosettes 223;
Step 2, a total volume change V_{Di} of the core 600 at the moment i is calculated according to the following formula:

$${V}_{Di} = {V}_{i} - Δ {V}_{F i} ;$$

wherein V_{Di} is the total volume change of the core 600 at the moment i; and Vᵢ is the liquid inlet amount of the annular pressure inlet hole 214 at the moment i; and the
amount of expansion deformation ΔV_{Fi} of the gripper system 200 at the moment i is calculated by using the following formula:

$$Δ {V}_{Fi} = {\frac{1}{64 π} [ {\sum }_{k = 1}^{4} {R}_{ki} \cdot ( 1 + {L}_{0} ) ]}^{2} \cdot \frac{1}{3} {\sum }_{k = 1}^{3} {V}_{ki} \cdot ( 1 \to {V}_{0} ) - \frac{{L}_{0}^{2} {V}_{0}}{4 π} ;$$

wherein π is a ratio of a circumference of a circle to its diameter; Rₖᵢ and Vₖᵢ are a radial strain and an axial strain detected by a strain rosette k at the moment i, respectively; and L₀ and V₀ are a radial circumference and an axial height of the gripping container, respectively.

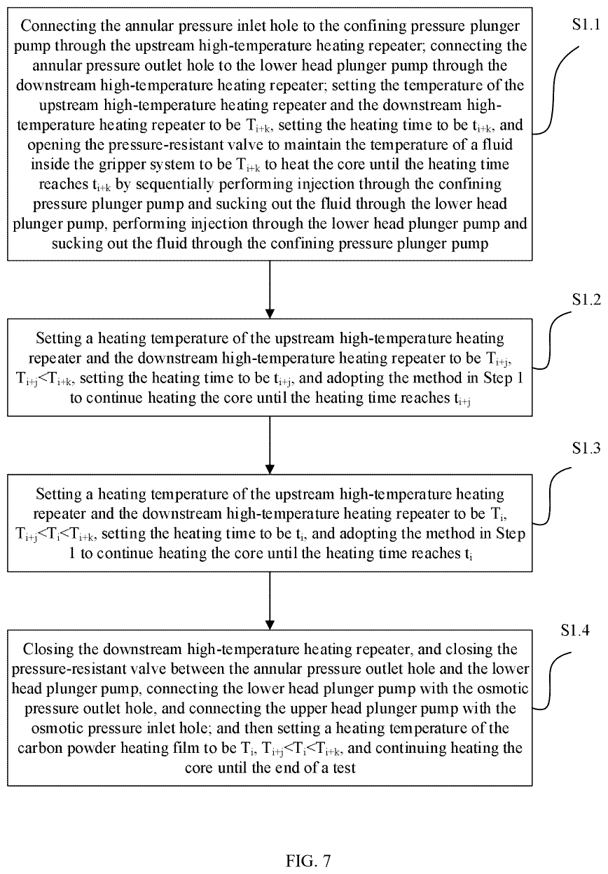

The core temperature field control method based on a temperature gradient as shown in FIG. 7 includes the steps of:
Step 1.1, connecting the annular pressure inlet hole 214 to the confining pressure plunger pump 410 through the upstream high-temperature heating repeater 420; connecting the annular pressure outlet hole 215 to the lower head plunger pump 450 through the downstream high-temperature heating repeater 430; setting the temperature of the upstream high-temperature heating repeater 420 and the downstream high-temperature heating repeater 430 to be Tᵢ₊ₖ, setting the heating time to be tᵢ₊ₖ, and opening the pressure-resistant valve 260 to maintain the temperature of a fluid inside the gripper system 200 to be Tᵢ₊ₖ to heat the core 600 until the heating time reaches tᵢ₊ₖ by sequentially performing injection through the confining pressure plunger pump 410 and sucking out the fluid through the lower head plunger pump 450, performing injection through the lower head plunger pump 450 and sucking out the fluid through the confining pressure plunger pump 410;
Step 1.2, setting a heating temperature of the upstream high-temperature heating repeater 420 and the downstream high-temperature heating repeater 430 to be Tᵢ₊ⱼ, Tᵢ₊ⱼ<Tᵢ₊ₖ, setting the heating time to be tᵢ₊ⱼ, and adopting the method in Step 1 to continue heating the core 600 until the heating time reaches tᵢ₊ⱼ;
Step 1.3, setting a heating temperature of the upstream high-temperature heating repeater 420 and the downstream high-temperature heating repeater 430 to be Tᵢ, Tᵢ₊ⱼ<Tᵢ<Tᵢ₊ₖ, setting the heating time to be tᵢ, and adopting the method in Step 1 to continue heating the core 600 until the heating time reaches tᵢ; and
Step 1.4, closing the downstream high-temperature heating repeater 430, and closing the pressure-resistant valve 260 between the annular pressure outlet hole 215 and the lower head plunger pump 450, connecting the lower head plunger pump 450 with the osmotic pressure outlet hole 245, and connecting the upper head plunger pump 440 with the osmotic pressure inlet hole 211; and then setting a heating temperature of the carbon powder heating film 224 to be Tᵢ, Tᵢ₊ⱼ<Tᵢ<Tᵢ₊ₖ, and continuing heating the core 600 until the end of a test.

The high-temperature and high-pressure core displacement test system and method according to the embodiments of the present application performs a displacement test by arranging the base 210, the gripping container 220 and the end cover 230 which are connected in sequence to enclose the core gripping cavity 250 to contain the core gripping module 240, and the alloy flange ends 222 are bonded to both ends of the carbon fiber wound PEEK sleeve 221 by using high-strength high-temperature-resistant ionic glue as the gripping container 220. The CT ray projection performance of the CT scanner 100 at high temperature and high pressure can be improved by using the carbon fiber wound PEEK sleeve 221, and the service life of the carbon fiber wound PEEK sleeve 221 can be prolonged by using the alloy flange ends 222. The stiffness-limited optical fiber 243 is arranged on the surface of the cylindrical core 600 to detect the surface deformation of the core 600, the optical fiber positioning precision is improved by the optical fiber limited deformation, and the deformation measurement precision of the core 600 in a fast scanning state of the CT scanner 100 is improved, and the overall volume change of the core 600 is corrected by measuring the expansion volume change of the gripper system 200, and the temperature rising speed of the core 600 and the sample temperature uniformity are improved by the gradient temperature control method, thereby enabling the core displacement CT test to be performed quickly and accurately under high temperature and high pressure conditions.

The embodiments described above are some embodiments of the present application, but not all of the embodiments. The detailed description of the embodiments of the application is not intended to limit the scope of the application, as claimed, but is merely representative of the selected embodiments of the application. Based on the embodiments in the present application, all other embodiments obtained by those of ordinary skill in the art without inventive step belong to the scope of protection of the present application.

What is claimed is:

1. A high-temperature and high-pressure core displacement test method, wherein the test method is performed using a high-temperature and high-pressure core displacement test system comprising:
a computed tomography (CT) scanner, configured to perform CT scanning;
a gripper system, comprising a base, a gripping container and an end cover which are connected in sequence, the base, the gripping container and the end cover enclosing a core gripping cavity containing a core gripping module, wherein the base is provided with an osmotic pressure inlet hole, an optical fiber inlet hole, an optical fiber outlet hole, and an annular pressure inlet hole and an annular pressure outlet hole which respectively communicate with the core gripping cavity; the gripping container comprises a carbon fiber wound polyether-ether-ketone (PEEK) sleeve sleeving the core gripping module, alloy flange ends respectively bonded to both ends of the carbon fiber wound PEEK sleeve, a plurality of strain rosettes disposed on an outer wall of the carbon fiber wound PEEK sleeve, and a carbon powder heating film sleeving outer sides of the strain rosettes, and the two alloy flange ends are respectively connected with the base and the end cover; the core gripping module comprises water permeable plates respectively clamped to two ends of a cylindrical core, a sample cap pressing against the end cover, a stiffness-limited optical fiber wound around an outer wall of the core, and a heat shrinkable sleeve sleeving an outer side of the stiffness-limited optical fiber, the two water permeable plates respectively abut against the base and the sample cap, the sample cap is provided with an osmotic pressure outlet hole, the osmotic pressure inlet hole and the osmotic pressure outlet hole respectively communicate with the two water permeable plates, and two ends of the stiffness-limited optical fiber respectively penetrate through the optical fiber inlet hole and the optical fiber outlet hole; and a pressure-resistant valve is connected to the annular pressure outlet hole;
a machine tool, configured to move the gripper system to the CT scanner for CT scanning of a core or move the gripper system out of the CT scanner;
a gripper load control system, configured to heat and input a fluid into the osmotic pressure inlet hole and the annular pressure inlet hole and receive a fluid output from the osmotic pressure outlet hole; and
an acquisition and data analysis system, configured to acquire a temperature and a pressure within the core gripping cavity, and detection data of the CT scanner and the strain rosettes for analysis; and
wherein the test method comprises the following steps:
step 1, controlling the gripper load control system to input a heating fluid into the osmotic pressure inlet hole, closing the pressure-resistant valve when a fluid continuously flows out of the annular pressure outlet hole, and adopting a core temperature field control method based on a temperature gradient to circulate a fluid inside the core gripping cavity to uniformly distribute the temperature of a core; and
step 2, calculating a total volume change V_{Di} of the core at a moment i according to the following formula:
$${V}_{Di} = {V}_{i} - Δ {V}_{F i} ;$$
wherein V_{Di} is the total volume change of the core at the moment i; and Vᵢ is a liquid inlet amount of the annular pressure inlet hole at the moment i; and calculating the amount of expansion deformation ΔV_{Fi} of the gripper system at the moment i by using the following formula:
$$Δ {V}_{Fi} = {\frac{1}{64 π} [ {\sum }_{k = 1}^{4} {R}_{ki} \cdot ( 1 + {L}_{0} ) ]}^{2} \cdot \frac{1}{3} {\sum }_{k = 1}^{3} {V}_{ki} \cdot ( 1 \to {V}_{0} ) - \frac{{L}_{0}^{2} {V}_{0}}{4 π} ;$$
wherein π is a ratio of a circumference of a circle to its diameter; Rₖᵢ and Vₖᵢ are a radial strain and an axial strain detected by a strain rosette k at the moment i, respectively; and L₀ and V₀ are a radial circumference and an axial height of the gripping container, respectively.

2. The high-temperature and high-pressure core displacement test method according to claim 1, wherein the base comprises a first step, a second step, a third step and a fourth step which are connected in sequence, the annular pressure inlet hole and the annular pressure outlet hole penetrate through the first step, the second step and the third step, and the fourth step abuts against the water permeable plate; and a peripheral wall of the third step and a peripheral wall of the fourth step are each provided with at least one perfluoroether high-temperature-resistant sealing ring.

3. The high-temperature and high-pressure core displacement test method according to claim 1, wherein the annular pressure inlet hole is connected with a pressure-resistant tube located inside the core gripping cavity, and a length of the pressure-resistant tube is 0.6-0.9 times a height of the core.

4. The high-temperature and high-pressure core displacement test method according to claim 1, wherein the sample cap comprises a fifth step and a sixth step which are connected in sequence, the fifth step presses against the water permeable plate, and a peripheral wall of the sixth step is provided with at least one perfluoroether high-temperature-resistant sealing ring.

5. The high-temperature and high-pressure core displacement test method according to claim 1, wherein the stiffness-limited optical fiber comprises a bare optical fiber and optical fiber sleeves nested and fixed to the bare optical fiber at intervals, a spacing between centers of two of the optical fiber sleeves being 0.65 D, D being a positive integer.

6. The high-temperature and high-pressure core displacement test method according to claim 1, wherein a resistance wire of each strain rosette is made of silicon; and data cables in the strain rosettes are composed of thin film graphene or black phosphorene strips, and the graphene or black phosphorene strips have a width of 0.25-0.26 mm and a thickness of 0.8-1.2 mm.

7. The high-temperature and high-pressure core displacement test method according to claim 1, wherein the gripper load control system comprises a confining pressure plunger pump connected to the annular pressure inlet hole, an upstream high-temperature heating repeater connected to the osmotic pressure inlet hole and a downstream high-temperature heating repeater connected to the osmotic pressure outlet hole, the upstream high-temperature heating repeater is connected with an upper head plunger pump, and the downstream high-temperature heating repeater is connected with a lower head plunger pump.

8. The high-temperature and high-pressure core displacement test method according to claim 1, further comprising a heat-insulating jacket wrapping the base, the gripping container, and the end cover.

9. The high-temperature and high-pressure core displacement test method according to claim 1, wherein the core temperature field control method based on the temperature gradient comprises the steps of:
step 1.1, connecting the annular pressure inlet hole to the confining pressure plunger pump through the upstream high-temperature heating repeater; connecting the annular pressure outlet hole to the lower head plunger pump through the downstream high-temperature heating repeater; setting the temperature of the upstream high-temperature heating repeater and the downstream high-temperature heating repeater to be Tᵢ₊ₖ, setting the heating time to be tᵢ₊ₖ, and opening the pressure-resistant valve to maintain the temperature of a fluid inside the gripper system to be Tᵢ₊ₖ to heat the core until the heating time reaches tᵢ₊ₖ by sequentially performing injection through the confining pressure plunger pump and sucking out the fluid through the lower head plunger pump, performing injection through the lower head plunger pump and sucking out the fluid through the confining pressure plunger pump;
step 1.2, setting a heating temperature of the upstream high-temperature heating repeater and the downstream high-temperature heating repeater to be Tᵢ₊ⱼ, Tᵢ₊ⱼ<Tᵢ₊ₖ, setting the heating time to be tᵢ₊ⱼ, and adopting the method in Step 1 to continue heating the core until the heating time reaches tᵢ₊ⱼ;
step 1.3, setting a heating temperature of the upstream high-temperature heating repeater and the downstream high-temperature heating repeater to be Tᵢ, Tᵢ₊ⱼ<Tᵢ<Tᵢ₊ₖ, setting the heating time to be tᵢ, and adopting the method in Step 1 to continue heating the core until the heating time reaches tᵢ; and
step 1.4, closing the downstream high-temperature heating repeater, and closing the pressure-resistant valve between the annular pressure outlet hole and the lower head plunger pump, connecting the lower head plunger pump with the osmotic pressure outlet hole, and connecting the upper head plunger pump with the osmotic pressure inlet hole; and then setting a heating temperature of the carbon powder heating film to be Tᵢ, Tᵢ₊ⱼ<Tᵢ<Tᵢ₊ₖ, and continuing heating the core until the end of a test.
